(19) United States
(12) Patent Application Publication
(10) Pub. No.: US 2013/0073018 A1
(43) Pub. Date: Mar. 21, 2013

(54) Thermal exchange composition-containing packs with color signaling covers

(71) Applicants: Gregg Harwood, Springfield, IL (US); Allen Morrow, Sherman, IL (US); Neil Puhse, Springfield, IL (US)

(72) Inventors: Gregg Harwood, Springfield, IL (US); Allen Morrow, Sherman, IL (US); Neil Puhse, Springfield, IL (US)

(21) Appl. No.: 13/373,171

(22) Filed: Nov. 7, 2011

Related U.S. Application Data
(60) Provisional application No. 61/535,557, filed on Sep. 16, 2011.

(51) Int. Cl. A61F 7/00

(52) U.S. Cl. 607/114

(57) ABSTRACT

The present invention relates to thermal exchange products having color change properties. More particularly, the invention relates to thermal-exchange compositions in pack form suitable for heating in a microwave oven and/or cooling in a freezer and having covers that provide a notification by color change signal that the pack is at a temperature or in a temperature range suitable to provide a desired level of hot and/or cold treatment.

### PRIORITY CLAIM

This application claims priority to U.S. Provisional Application No. 61/535,557, filed Sep. 16, 2011, which is incorporated herein in its entirety by this reference.

### FIELD OF THE INVENTION

The present invention relates to thermal exchange products having color change-properties. More particularly, the invention relates to thermal-exchange composition containing materials in pack form suitable for heating in a microwave oven and/or cooling in a freezer and having covers or portions of covers that provide a notification by color change signal that the pack is at a temperature or in a temperature range suitable to provide a desired level of hot and/or cold treatment.

### BACKGROUND OF THE INVENTION

Heat therapy is applied in a wide variety of contexts to reduce injury and to aid in recovery after exertions, injuries, and medical procedures. For example, heat therapy is often used for chronic conditions to help relax and loosen tissues, and to stimulate blood flow to the area. Heat treatments are also used for chronic conditions, such as overuse injuries, and before participating in activities. A variety of electrical heating pads are known for providing heat therapy that is linked to a power cord that plugs into a wall outlet. However, such electrical pads are often undesirable because they are intimately linked to the location of an electrical outlet.

In recent years, therapeutic packs comprising thermal exchange compositions have gained in popularity for the treatment of pain. In use, these packs can be heated in a microwave and/or cooled in a freezer and applied to provide the desired therapeutic effect while still allowing the user to remain fairly mobile during treatment. Moreover, these products are also reusable, thus making these products more cost-effective than one-time use heating or cooling products. Such products are also more environmentally conscious due to their multi-use qualities.

One widely used form of such thermal exchange composition-containing pack is manufactured by Thermionics Corporation (Springfield, Ill.) and sold as ThermiPaq®. As described in U.S. Pat. No. 5,478,988, the disclosure of which is incorporated herein in its entirety by this reference, this product can comprise a microwave reactive clay and a glycol, wherein the clay is present in the composition in an amount from about 20 percent to about 90 percent by weight and the glycol is present in the composition from about 10 percent to about 80 percent by weight. This product can be repeatedly heated and/or cooled by a user to provide a durable, yet portable, method to treat pain.

Other forms of reusable packs comprising thermal exchange compositions are in use such as gels or beads. Examples of a reuseable gel that can be heated in the microwave or cooled are ThermaKool® (Nortech Labs, Farmington, N.Y.). Silica gel beads or glycerin beads can also be used as thermal exchange compositions. Bead-containing reusable packs that are intended to be microwaved are made Thermionics Corporation and sold as ThermiBeads®. Yet further, thermal exchange compositions can comprise grain or herb filled bags.

Thermal exchange composition-containing packs such as those described above comprising microwaveable thermal exchange compositions are highly effective to provide heat and/or cold therapy, as well as general heating and cooling effects. For heat therapy, the consumer in need of therapeutic treatment need only place the pack in a microwave oven for the time and the power level recommended by the manufacturer to provide a hot pack for use. However, microwave ovens in use today have a wide range of operating parameters. As a result, even if the manufacturer provides a range of power levels and heating times, it is possible that the specific microwave oven being used will not heat the pack at the levels needed for therapeutic treatment to be effective. It is also possible that a user's microwave oven may overheat the pack, thereby putting the user in danger of burns, or subjecting the pack itself to deterioration and possible breakage. It would be helpful for the pack to allow the user to obtain the right temperature and to stay below the conditions that could cause damage to the person or the pack itself.

For cold treatment, a user must keep the pack at a cold temperature for a period sufficient to allow the pack to reach the therapeutic range. Often, however, the user will need the pack quickly after being injured, but will not have a pack waiting for use in their freezer. It would be helpful for the pack to allow the user to obtain the right temperature in the shortest possible time.

For both hot and cold therapeutic treatments, doctors frequently provide express instructions for the duration of treatment, and to ensure compliance, the patient should be able to know that the pack remained in the therapeutic temperature range as recommended. The hot pack may still feel hot to the touch of the user, but be outside of the effective therapeutic range for heat treatment. Similarly, the cold pack may feel cold to the touch, but be outside the effective therapeutic range for cold treatment.

In view of the above, it would be beneficial to have a thermal exchange composition-containing product that could provide a signal to the user providing information that the pack was at the appropriate temperature for use. Still further, it would be beneficial to provide a thermal exchange composition-containing product that provided information to the user that the pack was too hot for use.

### SUMMARY OF THE INVENTION

The present invention relates to hot and or cold releasing packs comprising thermal exchange compositions wherein the pack has a covering that provides a signal to the user when the pack is ready for use. Upon heating the pack to a desired temperature, particularly in a microwave oven, at least a portion of the cover changes color to notify the user that the pack has reached the desired temperature. The color change cover can have a baseline temperature color and at least one first temperature change color. The components of the cover are substantially transparent to microwave radiation or substantially resistant to self-heating in a microwave oven. Still further, the cover should provide a first visible color change at a pre-determined temperature thereby signaling to the user that the heating device is ready for use. In further aspects, the cover should substantially revert to the baseline color when the thermal exchange composition is substantially at the baseline temperature. The color change can occur at desired temperature for heating. Alternatively, the color change can occur at a suitable temperature for heating and remain at substantially the same color when the pack reaches and stays within a desired heating range. A further color change can occur when the pack gets “too hot.” The pack can also provide a suitable signal when cooled to a desired temperature.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 illustrates an aspect of the present invention where a thermal exchange composition-containing pack is covered by a reversible thermachromic cover comprising a patch of color change material.

FIG. 2 illustrates the invention of FIG. 1 wherein the patch of color change material has changed color to provide a signal to the user.

FIG. 3 illustrates an aspect of the present invention where a thermal exchange composition-containing pack is covered by a reversible thermachromic cover comprising in substantial entirety a color change material.

FIG. 4 illustrates an aspect of the present invention where a thermal exchange composition-containing pack is covered by a reversible thermachromic cover comprising color changing film in substantial entirety.

FIG. 5 illustrates an aspect of the present invention where a thermal exchange composition-containing pack has a thermochromic film patch affixed thereto.

### DETAILED DESCRIPTION OF THE INVENTION

The present invention may be understood more readily by reference to the following detailed description of preferred embodiments of the invention and the examples included therein and to the Figures and the previous and following description. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “thermal exchange composition” comprises a material or materials that have utility for use in heat and or cold therapy and are suitable for use multiple times. Such materials include, but are not limited to, clay-glycol (or other suitable hydroxyl-containing material such as glycerin), silica beads, ceramic beads, stone beads and silica-containing gels as discussed elsewhere herein. The specific type of thermal exchange composition utilized is not significant to the invention, as long as such compositions are suitable for multiple heating and cooling cycles. One time use compositions are not within the scope of the present invention. In this regard, heat therapy packs that work by providing an exothermic chemical reaction are not included herein. As such, the present invention does not relate to one time use products such as those disclosed in U.S. Patent Publication Nos. 2009/0149925 and 2009/0149772, the disclosures of which are incorporated herein in their entireties by this reference.

The present invention relates to hot and or cold releasing packs comprising thermal exchange compositions, as discussed further herein, wherein the pack has a covering that provides a signal to the user when the pack is ready for use. Upon heating of the pack to a desired temperature, particularly in a microwave oven, at least a portion of the cover changes color to notify the user that the pack has reached the desired temperature. In significant aspects, the desired temperature is in a range suitable to provide effective heat therapy. Still further, the color change of the cover can notify the user that the pack is too hot for use. In a separate aspect, upon being placed in a freezer for a suitable time, the color change of the cover can notify the user that the therapeutic pack is at a temperature suitable to provide effective cold therapy.

The thermal-exchange compositions of the present invention generally comprise a thermal exchange composition contained in an encapsulation material. The thermal exchange composition should be fully contained in the encapsulation material such that the composition does not leak out during each of the heating and/or cooling cycles or uses. The encapsulation material should therefore be a plastic-containing material that is suitable for multiple microwave heating and freezing cycles, such as discussed further herein. For bead-type therapies, the covering can comprise a fabric material.

The color change cover can have a baseline temperature color and at least one first temperature change color. The components of the cover should be substantially transparent to microwave radiation or substantially resistant to self-heating in a microwave oven. Still further, the cover should provide a first visible color change at a pre-determined temperature thereby signaling to the user that the heating device is ready for use. In further aspects, the cover should substantially revert to the baseline color when the thermal exchange composition is substantially at the baseline temperature.

In some aspects, the covering can provide a color change from one color to another to signal an appropriate temperature for heat therapy. In this regard, the cover can exhibit a color change when the pack temperature reaches about 110 or 120 or 130 or 140 or 150 or 160 or 165 degrees F. Still further, the covering can exhibit a color change at a particular temperature that corresponds to a desired heating temperature range and remain that color as long the pack stays within that specified range. For example, if the desired level of heating, such as for effective heat therapy, is from about 130 to about 165 degrees F., the cover can change color at or near 130 degrees F. and remain that same color (or substantially that same color) until the pack temperature exceeds about 165 degrees F. In yet a further aspect, when the pack exceeds the desired level of heating—here 165 degrees F. by way of example—the pack will change color again to signal to the user that the pack is “too hot” and should be allowed to cool before use.

In further aspects, the covering can provide a color change signal from one color to another to signal an appropriate temperature for applying a desired cold temperature, such as that suitable for cold therapy. In this regard, the cover can exhibit a color change when the pack temperature reaches about 35, 30 or 20 or 10 or 0 degrees F. Still further, the covering can exhibit a color change at a particular temperature that corresponds to a therapeutic temperature range and remain that color as long the pack stays within that specified range. For example, if the level for effective cold therapy is from about 35 to about 0 degrees F., the cover can change color at about 35 degrees F. and remain that same color (or substantially that same color) for lower temperatures. To signal that the pack has warmed beyond a desired range, for example, the desired range for cold therapy, the color can change again.

The color signals can be in the form of colors that would be viewed as appropriate to signal “hot” or “cold.” For example, if the pack is below the appropriate heat therapy temperature, the cover can remain the same color e.g. white or neutral, thus signifying “too cool.” When the pack reaches the therapeutic hot temperature or is in the therapeutic hot range, the cover could change to a red or pink hue to signal that it is “ready to use.” If the pack gets too hot, the cover could change to a black or the like. For cold therapy, the cover could change to from a neutral color to blue.

Alternatively, the color change signals could correspond to other colors, with the user being instructed of the significance of the respective colors using marketing collateral or by printed instructions on the cover itself. It is expected that after one or two uses, the user will understand the significance of the color signal to the desired hot or cold therapy.

To enhance the aesthetics of placing the thermal exchange composition-containing product on the skin surface of the user, the product may generally be fully covered with a fabric or the like. As such, the color changing cover can be constructed wholly from material that changes color. In other words, the entire cover can change color in use. Alternatively, the cover can be constructed in part from color change material—for example, the cover can have an area comprising color change material with the remainder of the cover being constructed from an aesthetically suitable fabric material. In this regard, the color change material can be sewn, laminated or glued onto the material from which the rest of the cover is constructed. The color change material can comprise one or more patches on the surface of a cover. The patch can be applied on top of such fabric or the fabric can be cut out in the approximate shape of the patch with the patch sewn, laminated or glued onto the cover.

In some aspects, the color change material can directly touch the thermal exchange composition in use. In further aspects, the color change composition can be separated from the thermal exchange composition by one or more layers. In this latter aspect, the separation will still suitably provide a color change signal to the user.

In use, it can be beneficial to place the color change material in close contact with the thermal exchange composition. When the color change material comprises a patch or the like, the user can be instructed to place the patch face down in the microwave (or in the freezer) to provide maximum contact with the thermal exchange composition. This aspect could enhance the accuracy of the color signaling of the present invention.

In further aspects, the color changes can also be presented in the form of patterns or wording on the cover. For example, the color signaling can be present on the cover in a form suitable to provide a visible “READY” or comparable statement upon when the pre-determined temperature is reached. This can be accomplished by incorporating the color change material into the cover in a pattern that conforms to the signaling words at the desired use temperature.

Further in regards to heat therapy, generally temperatures in the range of from about 135 degrees F. to about 170 degrees F. are found to be beneficial. However, the upper limit of this range might be too hot for some people, for example the elderly or children. In view of this, in some aspects, the reversible thermochromic cover of the present invention can signal to the user more than one range. For example, the reversible thermochromic covers of the present invention can allow the user to select a range of temperatures such as “low,” “medium,” or “high,” where such ranges can be signaled by a different color change in the reversible thermochromic fabric. In exemplary examples of this aspect, a low temperature range could be from about 110 to about 130 degrees F., a medium temperature range could be from about 140 to about 150 degrees F. and a high temperature range could be from about 150 to about 170 degrees F. Still further, these low, medium and high ranges could vary among these stated, as well as other, temperatures. Such variation would allow the user to customize his or her desired temperature.

In some aspects, a low temperature range would be that of a comfort level it would be good for relaxation and perhaps warm a person who is, cold while not necessarily providing heat at a therapeutic level. In further aspects, a medium temperature range would provide relaxation and some low grade therapy level. Such a setting would be especially helpful for people who consider the therapy level heat uncomfortable or have sensitive skin. Still further, the high temperature level could be within recommended therapy ranges.

Customization is also possible at cold therapy—otherwise known as “cyrotherapy”—treatment ranges. In some aspects, a color signal could be provided along the lines of “cold,” “colder” and “coldest.” Such levels can allow those with sensitive skin to avoid damage from application of packs that are too cold for their use. The reversible thermochromic fabric could also provide a signal of, for example, “cool” when the pack leaves hot therapy range.

For either or both of the hot or cold therapy, the reversible thermochromic covers could comprise multiple pieces or patches of color change fabric or other material or a single piece or patch of color change fabric material that is capable of providing color changes at various temperatures or ranges.

The packs of the present invention comprise thermal exchange compositions such as the clay-glycol (or glycerin), gels and beads discussed elsewhere herein. In significant aspects, the thermal exchange compositions comprising the packs of the present invention are microwave activatable clay compositions, gels or beads. The type of thermal exchange composition included in the pack is not significant to the invention as long as the composition incorporated in the pack is suitable for multiple heating and/or cooling cycles as described further herein

In further aspects, the color change signaling effects are provided by thermochromic materials that are incorporated on or in the covers to provide reversible color changing properties. As would be recognized by one of ordinary skill in the art, thermochromic materials predominantly change color in response to temperature fluctuations. There are two primary types of thermochromic materials: liquid crystals and leuco dyes. The thermochromic materials suitable for use in the present invention are reversible in that they not only change from a baseline temperature color when a pre-determined desired (trigger) temperature is reached, they change back to the baseline temperature color when the cover is again subjected to the baseline temperature. When reversible thermochromic materials are incorporated into fabrics or other materials that are, in turn, used in the construction of the covers of the present invention, the reversible thermochromic covers are provided.

The reversible thermochromic cover of the present invention can comprise a thermochromic ink, coating or the like coated onto non-woven fibers that are suitably transparent or substantially transparent to microwave energy. Such coated non-woven fabrics are disclosed in U.S. Pat. Nos. 6,228,804, 5,252,103, and 4,681,791, each of which are hereby incorporated by reference in their entireties by this reference. Alternatively, to provide the reversible thermochromic covers of the present invention, the thermochromic component can be incorporated within the fibers such as in the process described in U.S. Patent Publication No. 2003/0087566, which disclosure is incorporated by reference.

Still further, the reversible thermochromic covers of the present invention can comprise one or more film layers comprising thermochromic pigments laminated directly to the encapsulation material or laminated to a fabric, wherein the thermochromic pigments are incorporated during the film manufacturing process. In this aspect, the thermochromic pigments can be included during the manufacturing process by way of master batching or pre-coloring the resins. Such films, which, in some aspects, are believed to comprise low modulus polypropylene, polyethylene and thermoplastic elastomers are available from RTP Plastics (Winona, Minn.).

In specific aspects, these films can incorporated into fabric form by lamination of a backing material to the film. Thermochromic film-coated fabrics that can be manufactured into the reversible thermochromic covers of the present invention are available under the ChroMyx® brand name from Chameleon International (Oak Ridge, N.C.). Other suitable thermochromic pigment-containing film laminated fabrics that are believed suitable for manufacture into the reversible thermochromic covers of the present invention are disclosed in U.S. Pat. No. 5,928,803, which disclosure is incorporated by reference herein in its entirety.

Still further, the reversible thermochromic covers of the present invention can be prepared from thermochromic films comprising microencapsulated liquid crystal materials such as those supplied by LCR Hallcrest (Glenview, Ill.). These liquid crystal-containing films (as well as other suitable reversible thermochromic films or plastics) can be laminated or adhered to a number of fabric types that is placed on the pack, be laminated directly to the encapsulation material or formed into a plastic sleeve or cover that is placed on the pack.

For use in heat therapy, reversible thermochromic covers suitable for use in the present invention must be capable of repeated heating in a microwave oven at times and at temperature that will allow the thermal exchange composition-containing material to reach the appropriate heat therapy temperature. In this regard, the materials used to fabricate the reversible thermochromic covers of the present invention should not exhibit any appreciable melting or deterioration during multiple microwave heating operations suitable to provide enough heating to provide the desired heating effects of the associated thermal exchange composition-containing material. In other words, the reversible thermochromic covers of the present invention must be heat-resistant under the conditions reasonably expected by consumers who will use the product. The components comprising the reversible thermochromic covers of the present invention should therefore be “heat resistant.”

In this regard, “heat resistant” means that the reversible thermochromic cover of the present invention can be heated in a microwave for the 1 or 2 or 3 minutes at “high” temperature in a 700-1400 watt microwave for at least 3 or 4 or 5 or more times without exhibiting any appreciable deterioration of the components. “Without exhibiting any appreciable deterioration” means that the reversible thermochromic cover does not exhibit visible melting and/or loss of structural integrity that is manifested by tears, holes or fragility.

Significantly, many microwave ovens do not contain visible indication of the output wattage. In use, consumers may not know what the appropriate setting is for their particular device even if the thermal exchange composition-containing pack's packaging includes specific instructions regarding the suitable heating conditions for a range of microwave ovens. Moreover, the consumer may be confused by the relative size of their microwave ovens: some small ovens are on the high end of the wattage scale, and some large ovens are on the low end of the wattage scale. This dilemma is recognizable with microwave popcorn products in that manufacturers have included “popcorn settings” on microwave ovens for many years to prevent consumers from overheating popcorn where the microwave itself provides the “signal” that the popcorn is done by shutting off before too much energy is applied. However, for the thermal exchange composition-containing packs of the present invention, the “popcorn” (or other pre-sets that may be included by the manufacturer) are generally not suitable to provide the energy necessary to provide sufficient heating to the thermal exchange composition-containing products. The reversible thermochromic covers of the present invention meet the needs of the consumer to obtain the signal that the thermal exchange composition-containing product is ready for use.

Still further, the reversible thermochromic covers of the present invention should not exhibit significant deterioration in thermochromic color-changing characteristics for the duration of the useable life of the packs comprising thermal exchange compositions. In other words, the reversible thermochromic covers of the present invention should provide appropriate temperature signaling properties even after undergoing repeated heating in the types of microwave ovens in use by consumers of the product. This means that all aspects of the reversible thermochromic covers must be resistant to not only repeated heating cycles, but also to microwave radiation. In other words, the reversible thermochromic covers of the present invention must also be “microwave resistant.” In this regard, “microwave resistant” means that the reversible thermochromic cover of the present invention can be heated in a microwave for the 1 or 2 or 3 minutes at “high” temperature in an about 700, 800, 900, 1000, 1100, 1200, 1300 or 1400 watt microwave for at least 3 or 4 or 5 or more times without exhibiting any appreciable loss of thermochromic signaling properties. “Without exhibiting any appreciable loss in thermochromic signaling properties” means that the reversible thermochromic cover provides the appropriate signal to the user when the desired therapeutic heat level is reached after application of the microwave heating for the suitable time and level.

In addition to being both heat resistant and microwave resistant, the reversible thermochromic covers of the present invention should not become hot other than by heat transfer from the thermal exchange compositions. In other words, any color change signaling should be a result of the heating of the thermal exchange composition-containing material and not due to internal heating of the reversible thermochromic cover itself. As such, the reversible thermochromic cover—and the components that each, independently, comprise the finished reversible thermochromic cover should be substantially transparent to microwave radiation.

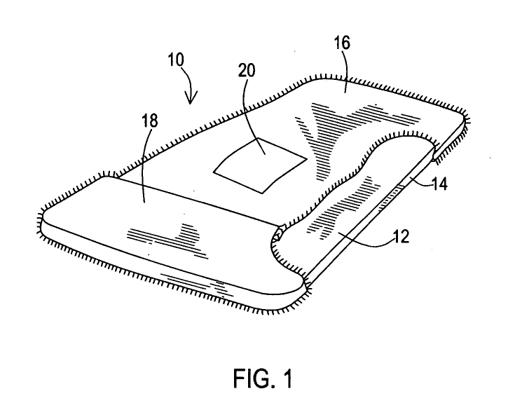

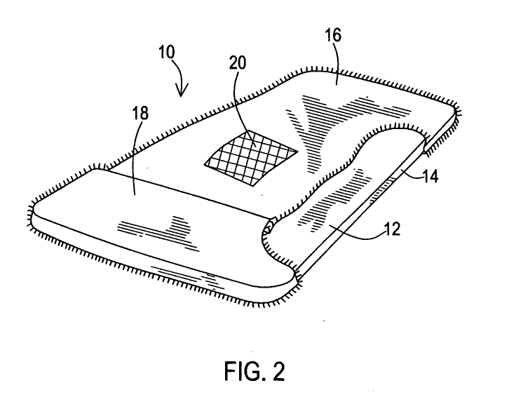

Referring now to the drawings, in FIG. 1 therapeutic pack 10 comprises a thermal exchange composition-containing pack 12 made from a plastic material 14 that encapsulates a thermal exchange composition (not shown). Covering the thermal exchange composition containing pack is fabric cover 16. A flap 18 can be included on cover 16 to allow the thermal exchange composition-containing pack to be inserted and removed. Reversible thermochromic material 20 is attached to cover 16 where the material 20 is suitable to change color to provide a signal to the user when pack 12 reaches a user temperature range. In FIG. 2, the reversible thermochromic material 20 has changed color to provide a signal to the user in response heating of the pack 10 in a microwave (not shown) to a use temperature range.

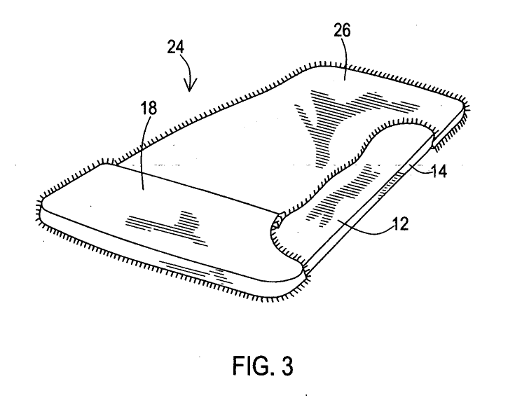

In FIG. 3, pack 12 has a fabric cover 26 comprising a reversible thermochromic material so that the entire cover 24 changes color in response to heating or cooling when it reaches a use temperature and then returns to a baseline color when it returns to a non-use temperature range.

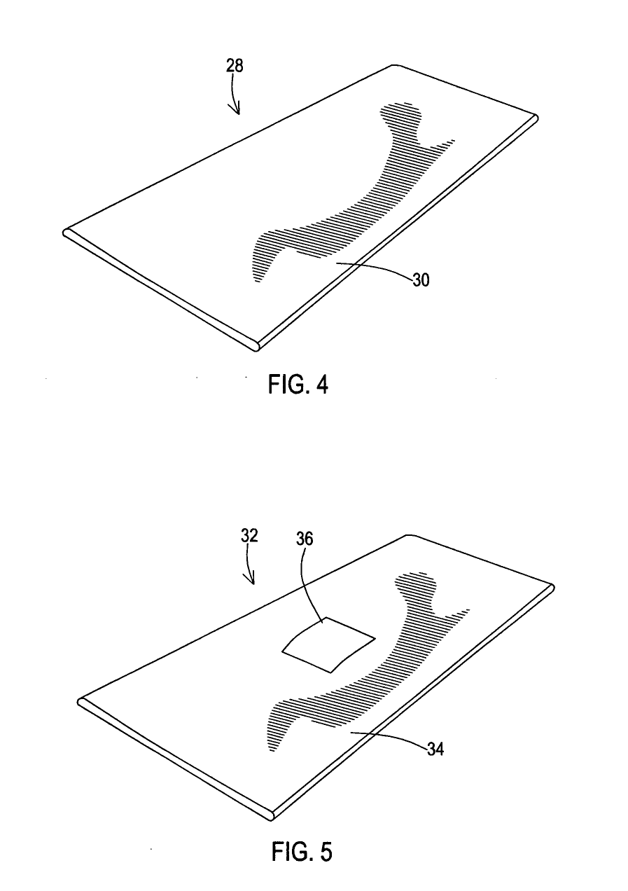

In FIG. 4, pack 28 comprises a thermal exchange composition containing pack (not shown) which is covered in its entirety with thermochromic film 30 so that the entire pack changes color in its entirety when heated or cooled to the use temperature range and then substantially reverts to a baseline color when it returns to a non-use temperature range.

In FIG. 5, pack 32 comprises a thermal exchange composition-containing pack 34 to which a patch of thermochromic film 36 is attached and can change color in response to heating or cooling to the use temperature range and then substantially reverts to a baseline color when it returns to a non-use temperature range.

### Experimental

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° F. or is at ambient temperature, and pressure is at or near atmospheric.

### Determination of Microwave-Resistance Characteristics of Reversible Thermochromic-Containing Covers

The reversible thermochromic fabric was a 2″×2″ piece of reversible thermochromic fabric sewn to a polyester fabric so that the thermochromic material was visible as a patch on the outside of the cover. The cover was placed in an 1100 W microwave oven for the specified time and observed for any color change and the observation temperatures recorded.

The reversible thermochromic fabric was a 2″×2″ piece of reversible thermochromic fabric sewn to a polyester fabric so that the thermochromic material was visible as a patch on the outside of the cover. The thermal exchange composition comprised a clay compound made from clay, glycol and water and comports to the material used in the commercial formulation sold as ThermiPaq® as described elsewhere herein. The material in the plastic pouch weighed approximately 1 pound, 8 ounces. The thermal exchange composition was encapsulated in a LLDPE material and was sealed with nylon. The encapsulated thermal exchange composition was placed in the cover with the thermochromic fabric side facing down to enhance contact of the fabric patch with the composition.

The fabric covered, encapsulated composition (i.e., pack) was placed in an 1100 W microwave oven for the specified time and observed for any color change and the observation temperatures recorded.

A series of sequential microwave heating and cooling cycles was conducted on the clay-containing packs covered with thermochromic film/laminate fabric. After heating, the appearance of the fabric and apparent temperature of the pack were observed.

| Heat time/Cycle # | Observations |
|---|---|
| 30 Seconds/1 time | Fabric color remained the same. The pack and fabrics warmed slightly |
| heating | |
| 30 Seconds/2 time | Fabric color remained the same. The pack and fabrics warmed slightly |
| heating | |
| 30 Seconds/3 time | Fabric color remained the same. The pack and fabrics warmed slightly |
| heating | |
| 30 Seconds/4 time | Fabric color remained the same. The pack and fabrics warmed slightly |
| heating | |
| 1 minute/5 time | Color changed slightly in a few locations. Pack and cover warmed to near |
| heating | desired temperature of at least about 130 degrees F. |
| 1 minute/6 time | Color changed slightly in a few locations. Pack and cover warmed to near |
| heating | desired temperature of at least about 130 degrees F. |
| 1 minute/7 time | Color changed slightly in a few locations. Pack and cover warmed to near |
| heating | desired temperature of at least about 130 degrees F. |
| 1 minute/8 time | Color changed slightly in a few locations. Pack and cover warmed to near |
| heating | therapy temperature. Color change material continues to return to original |
| | color as the clay cools. |
| 1 minute/9 time | Color changed slightly in a few locations. Pack and cover warmed to near |
| heating | therapy temperature. Color change material continues to return to original |
| | color as the clay cools. |
| 1 minute/10 time | Color changed slightly in a few locations. Pack and cover warmed to near |
| heating | therapy temperature. Color change material continues to return to original |
| | color as the clay cools. |

The above demonstrates that the thermochromic fabric was capable of being heated at least 10 times in microwave and still exhibiting reversibility. As such, the reversible thermochromic fabrics are microwave resistant and suitable for repeated heating by the user.

Determination of Temperature Characteristic of Thermal Exchange Composition-Containing Packs with Reversible Thermochromic Covers

Thermal exchange composition-containing packs covered by the reversible thermochromic covers were examined for time of heating in an 1100 watt microwave oven and the temperature reviewed to determine the signaling properties of the covers.

| Heat time | Observations |
|---|---|
| 1 min 20 sec/1 time | Full color Change Pack temp reached |
| heating | 150 degrees F. |
| 1 min 20 sec/2 time | Full color Change pack temp was 145 degrees F. |
| heating | |
| 1 min 20 sec/3 time | Full color Change pack temp was 148 degrees F. |
| heating | |
| 1 min 30 sec/4 time | Full color Change pack temp was 156 degrees F. |
| heating | |
| 1 min 25 sec/5 time | Full color Change pack temp was 157 degrees F. |
| heating | color change material continues to return to |
| | original color upon cooling |

The above results demonstrate that the reversible thermochromic covers of the present invention provide a signal to the user in the relevant temperature range for heat therapy.

The present invention is not limited in scope by the embodiments disclosed herein which are intended as illustrations of a few aspects of the invention and any embodiments which are functionally equivalent are within the scope of this invention. Various modifications of the apparatus and methods in addition to those shown and described herein will become apparent to those skilled in the art and are intended to fall within the scope of the appended claims. Further, while only certain representative combinations of the apparatus and method steps disclosed herein are specifically discussed in the embodiments above, other combinations of the apparatus components and method steps will become apparent to those skilled in the art and also are intended to fall within the scope of the appended claims. Thus a combination of components or steps may be explicitly mentioned herein; however, other combinations of components and steps are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

What is claimed is:

1) A product for applying a desired temperature to a user comprising:
a. a thermal exchange composition contained in an encapsulation material; and
b. a cover having a baseline color at a non-use temperature range and at least one first signal color at a first use temperature range,
wherein the cover consists essentially of components that are substantially transparent to microwave radiation,
wherein the cover changes to the first signal color when the thermal exchange composition reaches the first use temperature range,
and wherein the cover substantially reverts to the baseline color when the thermal exchange composition returns substantially to the non-use temperature range.

2) The product of claim 1 wherein the thermal exchange composition comprises microwave activatable clay compositions, ceramic beads or a gel.

3) The product of claim 1, wherein a lower limit of the first use temperature range is about 130 degrees F.

4) The product of claim 1, wherein an upper limit of the first use temperature is about 35 degrees F.

5) The product of claim 1, further comprising a second use temperature range of from about 0 to about 35 degrees F., wherein the cover provides a second signal color when the thermal exchange composition reaches the second use temperature range.

6) The product of claim 1, wherein the first use temperature range is from about 130 degrees F. to about 170 degrees F., wherein the cover remains a substantially the first signal color while the thermal exchange composition remains at the first use temperature range.

7) The product of claim 1, further comprising a first high temperature signal color, wherein the cover changes to the first high temperature signal color when the thermal exchange composition exceeds about 170 degrees F.

8) The product of claim 1, wherein the cover comprises a film comprising thermochromic pigments, wherein the film is laminated or glued to a fabric material.

9) The product of claim 1, wherein the cover comprises a patch comprising thermochromic pigments, wherein the patch is glued to an outer surface of the pack.
